(19) United States
(12) Patent Application Publication
(10) Pub. No.: US 2026/0245670 A1
(43) Pub. Date: Aug. 20, 2026

(54) DIFFUSIVE AUTOREGRESSION MODEL FOR GENERATING CHEMICAL STRUCTURES

(71) Applicant: Microsoft Technology Licensing, LLC, Redmond, WA (US)

(72) Inventors: Austin Henry CHENG, Vienna, VA (US); Alexis Woodward MILLS, Bellevue, WA (US); Hongbin LIU, Redmond, WA (US); Chong SUN, Austin, TX (US)

(73) Assignee: Microsoft Technology Licensing, LLC, Redmond, WA (US)

(21) Appl. No.: 19/057,809

(22) Filed: Feb. 19, 2025

(51) Int. Cl. G16C 20/50; G16C 20/70

(52) U.S. Cl. CPC G16C 20/50; G16C 20/70

(57) ABSTRACT

Examples are disclosed that relate to the use of diffusive autoregression models for generating 3-dimensional (3D) structures of chemical objects. One example provides a method, comprising a) inputting 3D structure data for a chemical object into a trained autoregression transformer model, b) receiving output from the trained autoregression transformer model, the output comprising encoding for a discrete atom type of a predicted next atom to be added to the molecule, c) inputting the 3D structure data for the chemical object and the discrete atom type into a trained diffusion model, d) receiving a position of the predicted next atom from the trained diffusion model, and e) updating the 3D structure data to include the position of the predicted next atom. The method further comprises iterating a), b), c), d), and e) until reaching a stopping criterion and outputting the 3D structure data for the candidate chemical object.

### BACKGROUND

In the field of chemical discovery, various computerized methods can be used to search for novel molecular and material structures for an intended application. Generative models are a class of computational models that use artificial intelligence (AI) to predict ways of combining atoms and/or molecular structure fragments to create novel structures.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

Examples are disclosed that relate to the use of diffusive autoregression models for generating 3-dimensional (3D) structures of chemical objects. One example provides a method of generating candidate structures using a diffusive autoregression model. The method comprises a) inputting 3D structure data for a chemical object into a trained autoregression transformer model. The method further comprises b) receiving output from the trained autoregression transformer model, the output comprising encoding for a discrete atom type of a predicted next atom to be added to the molecule. The method further comprises c) inputting the 3D structure data for the chemical object and the discrete atom type into a trained diffusion model. The method further comprises d) receiving a position of the predicted next atom from the trained diffusion model. The method further comprises e) updating the 3D structure data to include the position of the predicted next atom. The method further comprises f) iterating a), b), c), d), and e) until reaching a stopping criterion, and outputting the 3D structure data for the candidate chemical object.

### BRIEF DESCRIPTION OF THE DRAWINGS

FIG. 1 schematically shows an example of a computing architecture for implementing a diffusive autoregression model for generating 3D structures of novel chemical objects.

FIG. 2 schematically shows an example architecture for a diffusive autoregression model for generating chemical objects.

FIG. 3 schematically shows an example architecture for training a diffusive autoregression model.

FIG. 4 shows a flow diagram of an example method for generating chemical objects.

FIG. 5 shows results of an example trained diffusive autoregression model compared to other generative models.

FIG. 6 shows a block diagram of an example computing system.

### DETAILED DESCRIPTION

As introduced above, artificial intelligence (AI) models can be used to generate novel chemical objects including molecules and solid-state materials. Examples of generative modeling include autoregression models and diffusion models. Autoregression models are trained to predict a next token based on preceding tokens. In the realm of molecular generation, autoregression models can generate chemical structures by predicting a next atom to be added to a partially formed chemical structure. As an example, an autoregression model can predict a next token in a SMILES (simplified molecular-input line-entry system) string. In this manner, a chemical structure can be generated by sequentially adding symbols to a text string. autoregression models also can be applied to textual representations of 3-dimensional (3D) structure data, such as an XYZ file.

However, autoregression models may be limited in generating 3D structures. As autoregression models use tokenization, the models are trained to generate discrete values for atom positions. Discrete value predictions typically result in poor quality 3D structures compared to other models that can generate position data in a continuous regime.

As mentioned above, diffusion models also can be utilized for generating novel molecular structures. A diffusion model is trained to learn a velocity field that takes samples from a noisy distribution to an ordered distribution matching a data set. In the context of molecular generation, a diffusion model can denoise atom positions to yield a molecular structure. Diffusion models have performed well when applied to 3D structure prediction tasks, including conformer search (see, e.g., Wang, Y., Elhag, A. A., Jaitly, N., Susskind, J. M., & Bautista, M. Á. Swallowing the Bitter Pill: Simplified Scalable Conformer Generation. In Forty-first International Conference on Machine Learning.), protein-ligand docking (see, e.g., Corso, G., Stärk, H., Jing, B., Barzilay, R., & Jaakkola, T. (2022). Diffdock: Diffusion steps, twists, and turns for molecular docking. arXiv preprint arXiv: 2210.01776.), and biomolecular assembly (see, e.g., Abramson, J., Adler, J., Dunger, J. et al. Accurate structure prediction of biomolecular interactions with AlphaFold 3. Nature 630, 493-500 (2024)).

While diffusion models can demonstrate good performance in applications such as conformer search or docking, diffusion models are limited in certain aspects. One limitation for traditional diffusion models is that the number of atoms in a structure is fixed. The fixed atom constraint can pose issues for design of new molecules in 3D space as the number of atoms is not initially known. In some examples, a “jump diffusion” model can be used where the model is trained to learn a rate of transition between different discrete states having different number of atoms. However, jump diffusion models add new dimensions during the diffusion process that results in higher complexity models. This can pose a barrier to scalability for the molecular generation methods. Further, when using jump diffusion models for molecular generation it is unclear how to “complete” a molecule, i.e., determining not to jump to a state with a greater number of atoms. Further still, there is generally a lack of efficient techniques for training jump diffusion models. Additionally, diffusion models are typically trained for specific tasks and may have narrow applicability. For example, generating an organic molecule, generating defects in a crystal, and adding a solvation shell to a molecule are tasks that may use a corresponding different diffusion model. As such, a diffusion model may not be broadly applicable to various molecular building tasks. Further, training a different diffusion model for each specific task may be inefficient.

Accordingly, examples are disclosed that relate to diffusive autoregression models for generating 3D structures of chemical objects, including molecules and solid-state materials. The disclosed examples can generate novel structures by iteratively adding new atoms via an autoregression transformer model and predicting position data via a diffusion model. Instead of discrete tokens, the autoregression transformer encodes interdependence of tokens and produces a vector that conditions a denoising diffusion network. Briefly, 3D structure data for a chemical object is input into a trained autoregression transformer model. The trained autoregression transformer model predicts a new atom to be added to the chemical object and a conditioning label for a position of the new atom. The output from the autoregression transformer model is input with the 3D structure data into a trained diffusion model. The trained diffusion model predicts a position of the new atom. The 3D structure data is updated to include the position and atom type of the new atom and fed into the autoregression transformer in a next iteration. In this manner, the disclosed examples turn an autoregression model into a series of small diffusion models that generates 3D structure data atom by atom.

The disclosed examples can provide a more flexible and broadly applicable model for molecular generation. For example, a trained diffusive autoregression model can be used for a variety of different tasks, such as generating an organic molecule, hydrogenating a molecule, and/or decorating a molecule with solvent molecules (e.g., water). Further examples include generating a solid-state material, representing aperiodic amorphous materials, generating defects in a solid-state material, and/or sampling from grand canonical ensembles. As such, the disclosed examples can provide for training and deployment of fewer models compared to other generative methods. This helps improve computing efficiency. In some examples, additional conditioning can be used to further condition the diffusive autoregression model for a desired generation task. For example, decorating a molecule with hydrogen atoms can be performed by conditioning the model to add hydrogen atoms and not other atom types.

Additionally, the example diffusive autoregression models disclosed herein can help provide better quality chemical structures than other models while avoiding pitfalls associated with diffusion models and/or autoregression models. By using a diffusion model to generate a continuous position of a newly added atom, the hybrid model can avoid discrete resolution which degrade quality. By using an autoregression transformer model to predict the next atom to be added, the hybrid model avoids the fixed number of atoms of traditional diffusion models and the complexity of jump diffusion models.

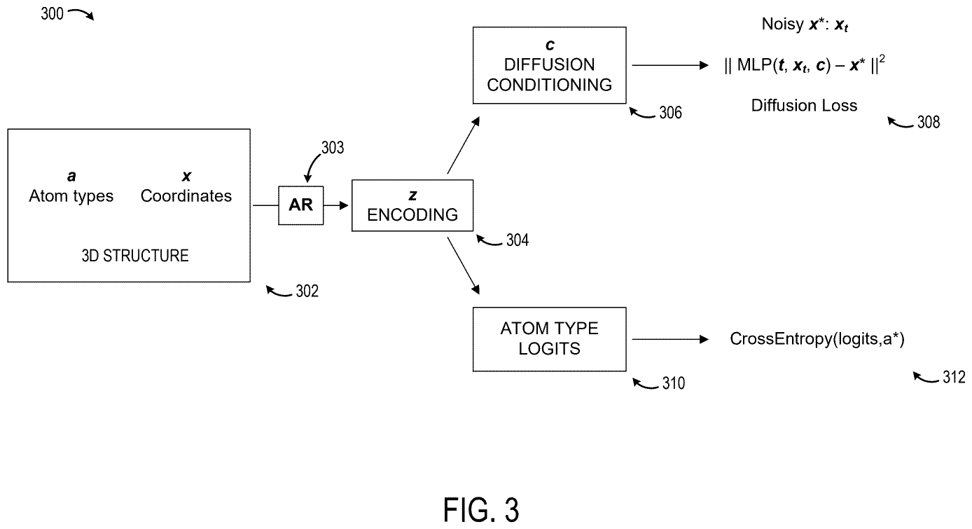

The diffusive autoregression model uses continuous-valued tokens as input and output. A discrete atom type of the next atom to be added can be determined using logits that operate on continuous-valued tokens and produce discrete values. Any suitable method can be used for training diffusive autoregression models. In some examples, the diffusion model is trained jointly with autoregression transformer model by backpropagation. Training is discussed in more detail below with regard to FIG. 3.

The autoregression model portion of the diffusive autoregression model can comprise a transformer, such as a Generative Pre-trained Transformer (GPT) model, as an example. Further, in some examples, the diffusive autoregression model can comprise a random-order autoregression model that predicts a next atom based on a random order of atoms of a partially completed chemical object. In some examples, the diffusive autoregression model can include a multilayer perceptron (MLP) as the diffusion model. MLPs are relatively smaller and easier to train compared to other relatively larger diffusion models. As such, use of a MLP can help reduce training time and/or reduce the complexity of the training process.

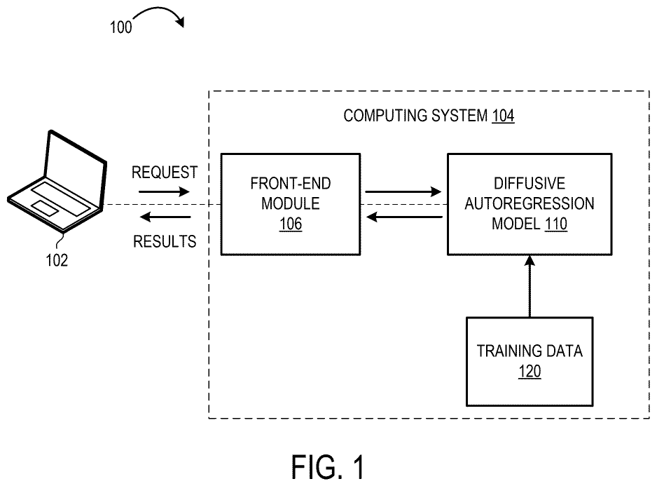

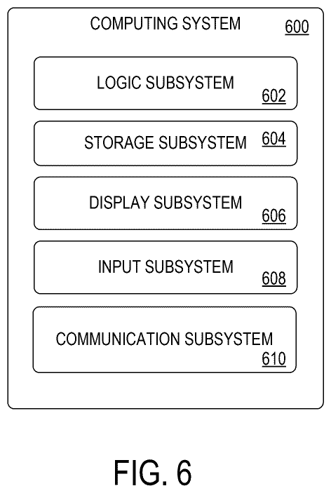

FIG. 1 schematically shows an example of a computing architecture 100 for processing a user request. As illustrated in FIG. 1, a client computer 102 can submit a user request to a computing system 104 implementing one or more diffusive autoregression models. Computing system 104 comprises one or more processors configured to process user requests, generate 3D structures of chemical objects, and perform various other functions. Computing system 104 can represent a data center in some examples. Examples of computing systems are described in more detail below with regard to FIG. 6.

Computing system 104 comprises a front-end module 106 for request processing. In some examples, front-end module 106 may be configured to capture natural language input (text or speech) from a user. Computing system 104 further may be configured to implement one or more natural language processing (NLP) models (e.g., a large language model (LLM)) to process data encoded in natural language input.

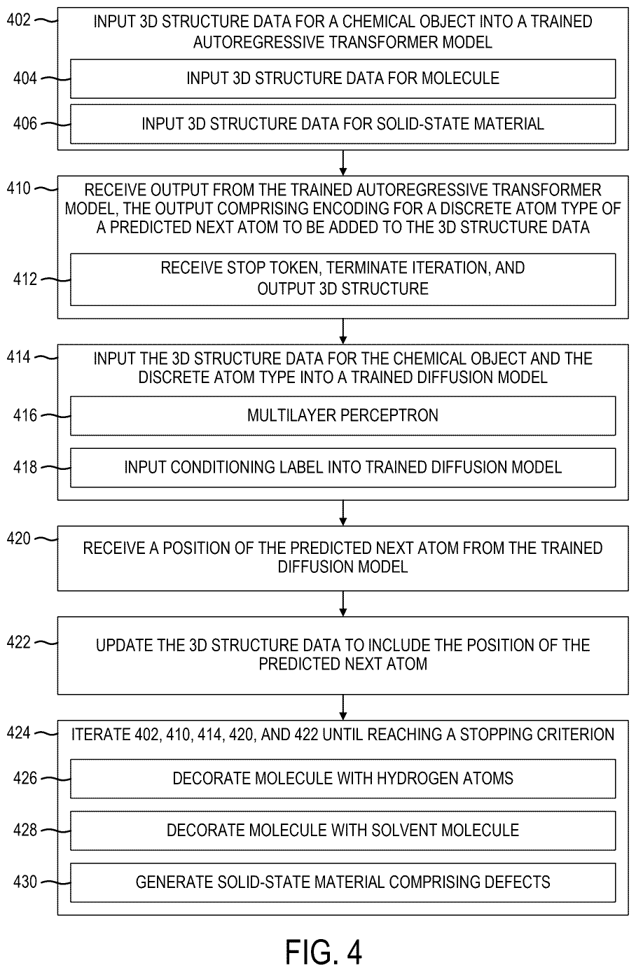

Computing system 104 further comprises a storage system storing data including data for one or more diffusive autoregression models 110 and optional training data 120. Upon receiving a user request, computing system 104 can use diffusive autoregression model 110 to generate one or more candidate chemical structures. Computing system 104 also is configured to output the one or more candidate chemical structures to client computer 102. The output candidate chemical structures comprise 3D structure data for chemical objects generated using diffusive autoregression model(s) 110. In some examples, a plurality of candidate chemical objects can be filtered (e.g., based on novelty) prior to outputting the candidate chemical objects to a user. An example method of generating 3D structures of chemical objects is discussed below with regard to FIG. 4.

In some examples, front-end module 106 can process user requests that include text-based conditioning for generating chemical objects. As one example, a user can provide 3D structure data for a molecule lacking hydrogen atoms with a request to add hydrogen atoms. Front-end module 106 can send the 3D structure data to diffusive autoregression model 110 with a text-based condition limiting the atom type of newly added atoms to “H”. As another example, a user request can include a request to “solvate a molecule”. In such an example, diffusive autoregression model 110 can be conditioned to add one or more solvent molecules to a 3D structure. In further examples, text-based conditioning can be used to specify other tasks, such as generating a solid-state material with defects or sampling a grand canonical ensemble.

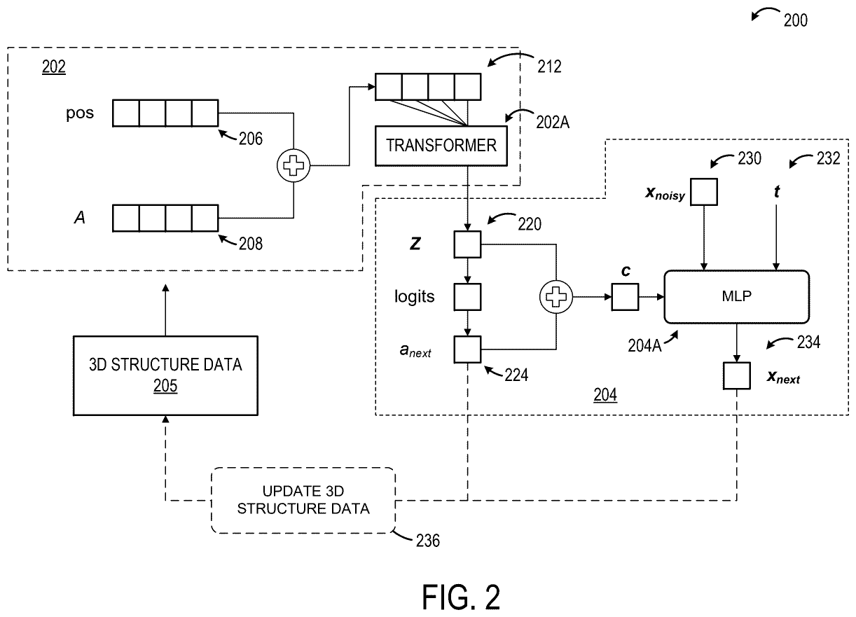

FIG. 2 shows an example architecture of a diffusive autoregression model 200 that can be used to generate 3D representations of chemical objects. Diffusive autoregression model 200 is an example of diffusive autoregression model 110 of FIG. 1. Diffusive autoregression model 200 comprises an autoregression transformer model 202 and a diffusion model 204. The diffusive autoregression model iteratively adds atoms to a chemical object (e.g., a molecular fragment) by predicting an atom type and position for a next atom. At each iteration, 3D structure data 205 for a chemical object is input into the diffusive autoregression model and the model produces an atom type for a next atom to be added to the structure.

Autoregression transformer model 202 can comprise any suitable model, such as a GPT model. Autoregression model 202 includes positional encoding for atom position data 206 and token embedding for atom type data 208. Atom type data 208 comprises discrete tokens associated with the element name of each atom (e.g., atomic number). Atomic position data 206 comprises continuous-valued tokens representing the 3D position of each corresponding atom (e.g., Cartesian coordinates). Atom type data and atom position data are combined in encoding block 212. At inference time, transformer 202A predicts a vector 220 (z) based on position data 206 and atom type data 208. Vector 220 is continuous-valued. To determine an atom type of the next atom to be added, logits 222 operate on vector 220 to output an atom type 224 (aₙₑₓₜ) of the next atom to be added to the structure. Atom type 224 is discrete-valued. As discussed below, logit operations on vector 220 also can produce a “stop” token. A stop token indicates to the diffusive autoregression model that the chemical structure generation is “complete” and no new atom is to be added.

Based on vector 220 and atom type 224, a conditioning vector 226 (c) is formed that conditions a MLP 204A for sampling a distribution for the 3D position of the next atom. Based on the conditioning vector 226, a noisy position 230 (x_{noisy}), and a noise time step 232 (t), MLP 204A produces a position 234 (xₙₑₓₜ) for the next atom to be added to the 3D structure data. Position 234 is a continuous-valued 3D position. A relatively smaller MLP can be easier to train compared to relatively larger diffusion models. In some examples, MLP 204A comprises four to ten layers. In some examples, MLP 204A comprises a width within a range of 1000 to 3000. In other examples, values outside these ranges can be used. As such, use of MLP 204A can help provide better efficiency than other methods. However, in other examples, any other suitable diffusion model can be used in place of, or in addition to, MLP 204A.

After predicting the atom type and position of the next atom, aₙₑₓₜ and xₙₑₓₜ are added to 3D structure data 205 to update the 3D structure data, as indicated at 236. Data for other atoms is not updated at 236 as MLP 204A does not move atoms other than the newly added atom. At the next iteration, updated 3D structure data 205 is used as input for the autoregression model 202. In some examples, a single atom is added to the 3D structure data per iteration. In some examples, a molecular fragment (e.g., a CH₃ group) or solvent molecule (e.g., water) can be added atom-by-atom.

Any suitable number of iterations can be performed to generate 3D structure data for the chemical object. Iterations proceed until a stop token is output. As mentioned above, when the chemical object is complete, transformer 202A can predict a stop token instead of a new atom type. The stop token indicates that no new atom is to be added to the chemical structure. Upon determining a stop token at 224 instead of a new atom type, 3D structure data 205 can be output as a “completed” chemical object.

The example diffusive autoregression model 200 of FIG. 2 does not explicitly include 3D symmetries. Instead, the model learns symmetries implicitly from random rotations and translations in training data. However, in other examples, a diffusive autoregression model can explicitly include 3D symmetries.

FIG. 3 shows an example architecture 300 for training a diffusive autoregression model, such as diffusive autoregression model 200. Training data includes a plurality of 3D structures 302 comprising atom types (a) and atom positions (x). Chemical objects 302 can include molecules, molecular fragments, and/or solid-state material structures. Training data can be obtained from any suitable source, including empirical data and theoretical data (e.g., 3D structures determined via quantum mechanics calculations).

As shown in FIG. 3, an autoregression model 303 learns to encode a vector (z) 304 comprising encoded information for the atom type and position of the next atom to be added to the chemical object. Vector 304 produces a conditioning vector (c) 306 for conditioning the diffusion model. This allows a probability distribution p(x|c) to be modeled for a position x of the next atom to be added conditioned on c. Vector 304 can comprise any suitable length, such as a length of 1000 to 3000. In other examples, a length outside this range can be used.

The training process uses a loss function 308 that serves as a denoising criterion. In the example shown, the loss function used is L(c, x)=∥MLP(t, xₜ, c)−x*∥², where MLP represents a multilayer perceptron, x* is a noisy position, and t is a noise schedule time step. Any suitable parameters can be used for the MLP. In some examples, the MLP comprises relatively few residual blocks (e.g., 2 to 10). In other examples, a greater number of residual blocks can be used. Blocks may comprise any suitable width, such as a width of 1000 to 3000 in some examples. In some examples, the diffusion process uses a noise schedule with a cosine shape and 100 to 5000 steps at training time. In other examples, a number of steps outside this range can be used. At inference time, the noise can be resampled with fewer steps, in some examples.

The autoregression model 303 also learns to output atom type logits 310 on vector 306. As shown in architecture 300, a cross-entropy loss function 312 is used to measure the atom type prediction performance of the autoregression model. In other examples, a different loss function can be used, such as a hinge loss. Loss function 308 and cross-entropy loss function 312 are used to jointly train the autoregression model and the diffusion model. Any suitable training method can be used, such as backpropagation. The training architecture and training objectives shown in FIG. 3 are relatively simple compared to other models, such as training a jump diffusion model. Due to simplicity, training the diffusive autoregression model can scale better than other more complex training architectures.

FIG. 4 shows a flow diagram of an example method 400 for generating candidate chemical object using a diffusive autoregression model. At 402, method 400 comprises inputting 3D structure data for a chemical object into a trained autoregression model. In some examples, at 404, the chemical object comprises a molecule. Alternatively or additionally, in some examples, at 406, the chemical object comprises a solid-state material.

Method 400 further comprises, at 410, receiving output from the trained autoregression transformer model. The output comprises encoding for a discrete atom type of a predicted next atom to be added to the 3D structure data. In some examples, the discrete atom type is determined by logits applied to a vector output from the trained autoregression transformer model. The output at 410 can further comprise encoding for a conditioning vector for conditioning a diffusion model. In some examples, at 412, the predicted atom type comprises a stop token indicating that no additional atoms are to be added to the 3D structure. Upon output of a stop token, method 400 can terminate iterations and output the 3D structure data of the current iteration.

Method 400 further comprises, at 414, inputting the 3D structure data for the chemical object and the discrete atom type into a trained diffusion model. As discussed above, the trained diffusion model is configured to denoise a noisy position of the predicted next atom based on a conditioning vector. In some examples, at 416, the diffusion model comprises a multilayer perceptron. In other examples, a different type of diffusion model can be used. In some examples, at 418, the method comprises inputting a conditioning label into the trained diffusion model, where single atom conditioning is completed on the next atom to be added and passed to the diffusion model.

Continuing, at 420, method 400 comprises receiving a position of the predicted next atom from the trained diffusion model. At 422, method 400 comprises updating the 3D structure data to include the position and atom type of the predicted next atom. The updated 3D structure data can be output upon determining a stop token at step 312.

As indicated at 424, method 400 further comprises iterating steps 402, 410, 414, 420, and 422 until reaching a stopping criterion. Any suitable stopping criterion can be used. As mentioned above at 414, output of a stop token as a predicted next atom type can serve as a stopping criterion indicating that the chemical object is complete and no new atoms are to be added. Thus, method 400 can terminate after receiving a stop token at 414. Further, in some examples, a stopping criterion can comprise reaching a selected threshold number of atoms. For example, the diffusive autoregression model can be used to add a selected number of solvent molecules to a target chemical object. As another example, sampling a grand canonical ensemble can involve generating a plurality of chemical objects each having a selected number of atoms.

In some examples, at 426, method 400 comprises decorating a molecule with hydrogen atoms. In some examples, at 428, method 400 comprises decorating a molecule with solvent molecules. In some examples, at 430, method 400 comprises generating a solid-state material comprising defects. In other examples, method 400 can be used to generate a different type of chemical object as requested by a user. As mentioned above, text-based conditioning can be used to request a specific task and/or limit atom types generated by the autoregression transformer model.

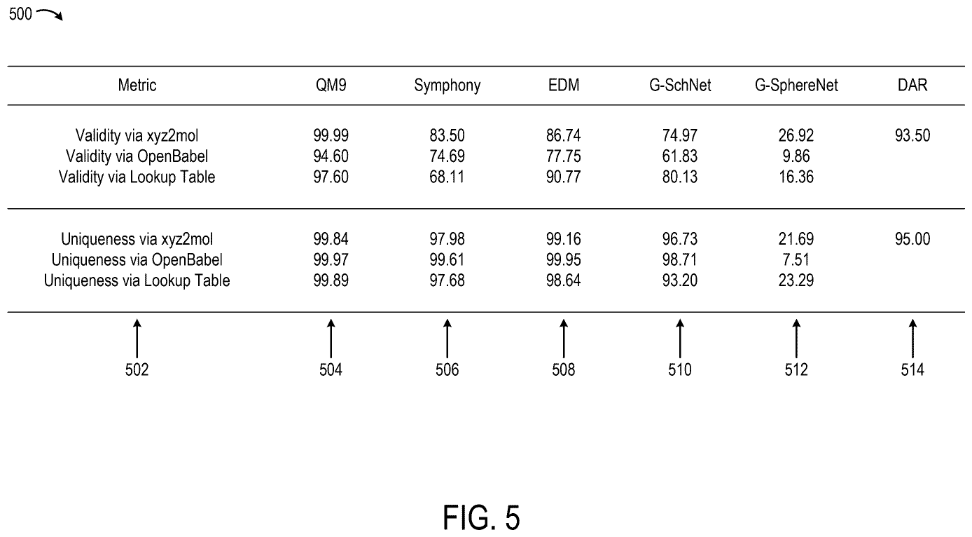

FIG. 5 shows a table 500 of experimental results of an example trained diffusive autoregression model compared to other generative models. In this experiment, the QM9 dataset (Ramakrishnan, R., Dral, P., Rupp, M. et al. Quantum chemistry structures and properties of 134 kilo molecules. Sci Data 1, 140022 (2014).) was used to train a diffusive autoregression model. The QM9 dataset also was used as a benchmark for evaluating the models. As shown in column 502 of table 500, the various models are evaluated based on validity and uniqueness. The validity score represents a percentage of generated molecules that contain correct bond orders and bond lengths without severe steric effects or overlapping atoms. The uniqueness score represents a percentage of generated molecules that are unique. Column 504 shows scores for the QM9 benchmark dataset.

Column 506 shows results for Symphony, an E(3)-equivariant autoregression model (Daigavane, A., Kim, S., Geiger, M., & Smidt, T. (2023). Symphony: Symmetry-Equivariant Point-Centered Spherical Harmonics for Molecule Generation. arXiv preprint arXiv: 2311.16199.). Column 508 shows results for EDM, a diffusion model (Hoogeboom, E., Satorras, V. G., Vignac, C., & Welling, M. (2022, Jun.). Equivariant diffusion for molecule generation in 3d. International conference on machine learning (pp. 8867-8887). PMLR.). Column 510 shows results for G-SchNet, an autoregression model (Gebauer, N. W., Gastegger, M., Hessmann, S. S., Müller, K. R., & Schütt, K. T. (2022). Inverse design of 3d molecular structures with conditional generative neural networks. Nature communications, 13(1), 973.). Column 512 shows results for G-SphereNet, an autoregression model (Youzhi Luo and Shuiwang Ji (2022). An Autoregressive Flow Model for 3D Molecular Geometry Generation from Scratch. International Conference on Learning Representations.).

Column 514 shows results for a diffusive autoregression (DAR) model trained using the example training methods described above. The diffusive autoregression was trained using the QM9 dataset. As shown in Table 500, the diffusive autoregression model of column 514 results in higher validity score than the other models reported in columns 506, 508, 510, 512.

The uniqueness scores of the diffusive autoregression model is slightly lower than the scores for Symphony and EDM. The slightly lower uniqueness of the diffusive autoregression model can potentially be addressed several ways. As the diffusive autoregression model of column 514 is a relatively large model trained on a relatively smaller dataset (approximately 134 k molecules), the model may suffer from “overfitting” where it generates the same molecule more than once. As such, it can help to train the model on a relatively larger dataset than QM9. Relatively larger datasets can help the model explore a more diverse set of chemical motifs. As another possibility, atom order can be perturbed. Adding perturbations to atom order generation at inference time may help to produce candidate chemical structures with greater uniqueness scores.

As another experiment, the diffusive autoregression model of column 514 was used to decorating a molecule with hydrogen atoms. In small molecule generative tasks, hydrogen atoms are typically added to a chemical structure at the end of structure generation. As such, 3D structures of small molecules lacking hydrogen atoms were used as input in the experiment. Further, the diffusive autoregression model was conditioned to add hydrogen atoms and not add other atom types. In the experiment, the diffusive autoregression model added the correct number of hydrogen atoms 95.2% of the time. Further, structure prediction was correct to within 0.5 Angstroms (Å) RMSD (root mean square deviation).

Thus, the disclosed examples provide for diffusive autoregression models for generating 3D structures of candidate chemical structures with better performance than other models. The example diffusive autoregression models can produce 3D structures with higher validity than other models. Additionally, the disclosed example models can be applied to a variety of generative tasks, including molecules, solid-state materials, hydrogen decoration, and solvation. A more flexible generative model can help improve efficiency in training and deployment phases. Further, the use of a hybrid model that combines diffusion and autoregression can help overcome limitations associated with either model alone, such as the fixed-number-of-atoms limitation of diffusion models and the discretized position limitation of autoregression models.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 6 schematically shows a non-limiting embodiment of a computing system 600 that can enact one or more of the methods and processes described above. Computing system 600 is shown in simplified form. Computing system 600 may take the form of one or more personal computers, server computers, tablet computers, home-entertainment computers, network computing devices, gaming devices, mobile computing devices, mobile communication devices (e.g., smart phone), and/or other computing devices.

Computing system 600 includes a logic subsystem 602 and a storage subsystem 604. Computing system 600 may optionally include a display subsystem 606, input subsystem 608, communication subsystem 608, and/or other components not shown in FIG. 6.

Logic subsystem 602 includes one or more physical devices configured to execute instructions. For example, the logic subsystem may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem may include one or more processors configured to execute software instructions. Additionally or alternatively, the logic subsystem may include one or more hardware or firmware logic subsystems configured to execute hardware or firmware instructions. Processors of the logic subsystem may be single-core or multi-core, and the instructions executed thereon may be configured for sequential, parallel, and/or distributed processing. Individual components of the logic subsystem optionally may be distributed among two or more separate devices, which may be remotely located and/or configured for coordinated processing. Aspects of the logic subsystem may be virtualized and executed by remotely accessible, networked computing devices configured in a cloud-computing configuration.

Storage subsystem 604 includes one or more physical devices configured to hold instructions executable by the logic subsystem to implement the methods and processes described herein. When such methods and processes are implemented, the state of storage subsystem 604 may be transformed—e.g., to hold different data.

Storage subsystem 604 may include removable and/or built-in devices. Storage subsystem 604 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage subsystem 604 may include volatile, nonvolatile, dynamic, static, read/write, read-only, random-access, sequential-access, location-addressable, file-addressable, and/or content-addressable devices.

It will be appreciated that storage subsystem 604 includes one or more physical devices. However, aspects of the instructions described herein alternatively may be propagated by a communication medium (e.g., an electromagnetic signal, an optical signal, etc.) that is not held by a physical device for a finite duration.

Aspects of logic subsystem 602 and storage subsystem 604 may be integrated together into one or more hardware-logic components. Such hardware-logic components may include field-programmable gate arrays (FPGAs), program-and application-specific integrated circuits (PASIC/ASICs), program-and application-specific standard products (PSSP/ASSPs), system-on-a-chip (SOC), and complex programmable logic devices (CPLDs), for example.

The terms “module,” “program,” and “engine” may be used to describe an aspect of computing system 600 implemented to perform a particular function. In some cases, a module, program, or engine may be instantiated via logic subsystem 602 executing instructions held by storage subsystem 604. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 606 may be used to present a visual representation of data held by storage subsystem 604. This visual representation may take the form of a graphical user interface (GUI). As the herein described methods and processes change the data held by the storage subsystem, and thus transform the state of the storage subsystem, the state of display subsystem 606 may likewise be transformed to visually represent changes in the underlying data. Display subsystem 606 may include one or more display devices utilizing virtually any type of technology. Such display devices may be combined with logic subsystem 602 and/or storage subsystem 604 in a shared enclosure, or such display devices may be peripheral display devices.

When included, input subsystem 608 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on-or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 608 may be configured to communicatively couple computing system 600 with one or more other computing devices. Communication subsystem 608 may include wired and/or wireless communication devices compatible with one or more different communication protocols. As non-limiting examples, the communication subsystem may be configured for communication via a wireless telephone network, or a wired or wireless local-or wide-area network. In some embodiments, the communication subsystem may allow computing system 600 to send and/or receive messages to and/or from other devices via a network such as the Internet.

Another example provides a method of generating candidate chemical structures using a diffusive autoregression model, the method comprising a) inputting 3-dimensional (3D) structure data for a chemical object into a trained autoregression transformer model, b) receiving output from the trained autoregression transformer model, the output comprising encoding for a discrete atom type of a predicted next atom to be added to the molecule, c) inputting the 3D structure data for the chemical object and the discrete atom type into a trained diffusion model, d) receiving a position of the predicted next atom from the trained diffusion model, e) updating the 3D structure data to include the position of the predicted next atom, f) iterating a), b), c), d), and e) until reaching a stopping criterion, and outputting the 3D structure data for the chemical object. In some such examples, the chemical object comprises a molecule. Alternatively or additionally, in some such examples, iterating a), b), c), d), and e) comprises decorating the molecule with one or more of hydrogen atoms or solvent molecules. Alternatively or additionally, in some such examples, the chemical object comprises a solid-state material. Alternatively or additionally, in some such examples, iterating a), b), c), d), and e) comprises generating defects in the solid-state material. Alternatively or additionally, in some such examples, inputting the 3D structure data for the chemical object and the discrete atom type into the trained diffusion model comprises inputting the 3D structure data for the chemical object and the discrete atom type into a multilayer perceptron. Alternatively or additionally, in some such examples, b) receiving output from the trained autoregression transformer model further comprises receiving a conditioning label for the position of the new atom, and c) inputting the 3D structure data for the chemical object and the discrete atom type into the trained diffusion model further comprises inputting the conditioning label into the trained diffusion model. Alternatively or additionally, in some such examples, b) receiving output from the trained autoregression transformer model comprises receiving one of a flag indicating the discrete atom type of the predicted next atom or a flag indicating the stopping criterion. Alternatively or additionally, in some such examples, the output from the trained autoregression transformer model comprises encoding for a conditioning vector for conditioning the trained diffusion model.

Another example provides a computing system implementing a diffusive autoregression model for generating candidate structures, the computing system comprising a logic subsystem and a storage subsystem. The storage subsystem stores instructions executable by the logic system to a) input 3-dimensional (3D) structure data for a chemical object into a trained autoregression transformer model, b) receive output from the trained autoregression transformer model, the output comprising a discrete atom type of a predicted next atom to be added to the molecule, c) input the 3D structure data for the chemical object and the discrete atom type into a trained diffusion model, d) receive a position of the predicted next atom from the trained diffusion model, e) update the 3D structure data to include the position of the predicted next atom, and output the 3D structure data. In some such examples, the instructions are further executable to iterate a), b), c), d), and e) prior to outputting the 3D structure data. In some such examples, the instructions executable to iterate a), b), c), d), and e) comprise instructions executable to decorate a molecule with one or more hydrogen atoms. Alternatively or additionally, in some such examples, the instructions executable to iterate a), b), c), d), and e) comprise instructions executable to decorate a molecule with one or more solvent molecules. Alternatively or additionally, in some such examples, the instructions executable to iterate a), b), c), d), and e) comprise instructions executable to generate defects in a solid-state material. Alternatively or additionally, in some such examples, the instructions are executable to input the 3D structure data for the chemical object and the discrete atom type into a multilayer perceptron. Alternatively or additionally, in some such examples, the multilayer perceptron comprises four to ten layers and a width within a range of 1000 to 3000.

Another example provides a method of generating candidate structures using a diffusive autoregression model, the method comprising a) inputting 3-dimensional (3D) structure data for a chemical object into a trained autoregression transformer model, b) receiving output from the trained autoregression transformer model, the output comprising a discrete atom type of a predicted next atom to be added to the molecule and a conditioning label for the position of the new atom, c) inputting the 3D structure data for the chemical object, the discrete atom type, and the conditioning label into a trained diffusion model, d) receiving a position of the predicted next atom from the trained diffusion model, and e) updating the 3D structure data to include the position of the predicted next atom. In some such examples, the method further comprises iterating a), b), c), d), and e) prior to outputting the 3D structure data. Alternatively or additionally, in some such examples, iterating a), b), c), d), and e) comprises decorating a molecule with one or more of hydrogen atoms or solvent molecules. Alternatively or additionally, in some such examples, inputting the 3D structure data for the chemical object, the discrete atom type, and the conditional label into the trained diffusion model comprises inputting the 3D structure data for the chemical object, the discrete atom type, and the conditioning label into a multilayer perceptron.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A method of generating candidate chemical structures using a diffusive autoregression model, the method comprising:
a) inputting 3-dimensional (3D) structure data for a chemical object into a trained autoregression transformer model;
b) receiving output from the trained autoregression transformer model, the output comprising encoding for a discrete atom type of a predicted next atom to be added to the molecule;
c) inputting the 3D structure data for the chemical object and the discrete atom type into a trained diffusion model;
d) receiving a position of the predicted next atom from the trained diffusion model;
e) updating the 3D structure data to include the position of the predicted next atom;
f) iterating a), b), c), d), and e) until reaching a stopping criterion; and
outputting the 3D structure data for the chemical object.

2. The method of claim 1, wherein the chemical object comprises a molecule.

3. The method of claim 2, wherein iterating a), b), c), d), and e) comprises decorating the molecule with one or more of hydrogen atoms or solvent molecules.

4. The method of claim 1, wherein the chemical object comprises a solid-state material.

5. The method of claim 4, wherein iterating a), b), c), d), and e) comprises generating defects in the solid-state material.

6. The method of claim 1, wherein inputting the 3D structure data for the chemical object and the discrete atom type into the trained diffusion model comprises inputting the 3D structure data for the chemical object and the discrete atom type into a multilayer perceptron.

7. The method of claim 1, wherein b) receiving output from the trained autoregression transformer model further comprises receiving a conditioning label for the position of the new atom, and wherein c) inputting the 3D structure data for the chemical object and the discrete atom type into the trained diffusion model further comprises inputting the conditioning label into the trained diffusion model.

8. The method of claim 1, wherein b) receiving output from the trained autoregression transformer model comprises receiving one of a flag indicating the discrete atom type of the predicted next atom or a flag indicating the stopping criterion.

9. The method of claim 1, wherein the output from the trained autoregression transformer model comprises encoding for a conditioning vector for conditioning the trained diffusion model.

10. A computing system implementing a diffusive autoregression model for generating candidate structures, the computing system comprising:
a logic subsystem; and
a storage subsystem storing instructions executable by the logic system to
a) input 3-dimensional (3D) structure data for a chemical object into a trained autoregression transformer model,
b) receive output from the trained autoregression transformer model, the output comprising a discrete atom type of a predicted next atom to be added to the molecule,
c) input the 3D structure data for the chemical object and the discrete atom type into a trained diffusion model,
d) receive a position of the predicted next atom from the trained diffusion model,
e) update the 3D structure data to include the position of the predicted next atom, and
output the 3D structure data.

11. The computing system of claim 10, wherein the instructions are further executable to iterate a), b), c), d), and e) prior to outputting the 3D structure data.

12. The computing system of claim 11, wherein the instructions executable to iterate a), b), c), d), and e) comprise instructions executable to decorate a molecule with one or more hydrogen atoms.

13. The computing system of claim 11, wherein the instructions executable to iterate a), b), c), d), and e) comprise instructions executable to decorate a molecule with one or more solvent molecules.

14. The computing system of claim 11, wherein the instructions executable to iterate a), b), c), d), and e) comprise instructions executable to generate defects in a solid-state material.

15. The computing system of claim 10, wherein the instructions are executable to input the 3D structure data for the chemical object and the discrete atom type into a multilayer perceptron.

16. The computing system of claim 15, wherein the multilayer perceptron comprises four to ten layers and a width within a range of 1000 to 3000.

17. A method of generating candidate structures using a diffusive autoregression model, the method comprising:
a) inputting 3-dimensional (3D) structure data for a chemical object into a trained autoregression transformer model;
b) receiving output from the trained autoregression transformer model, the output comprising a discrete atom type of a predicted next atom to be added to the molecule and a conditioning label for the position of the new atom;
c) inputting the 3D structure data for the chemical object, the discrete atom type, and the conditioning label into a trained diffusion model;
d) receiving a position of the predicted next atom from the trained diffusion model; and
e) updating the 3D structure data to include the position of the predicted next atom.

18. The method of claim 17, further comprising iterating a), b), c), d), and e) prior to outputting the 3D structure data.

19. The method of claim 18, wherein iterating a), b), c), d), and e) comprises decorating a molecule with one or more of hydrogen atoms or solvent molecules.

20. The method of claim 17, wherein inputting the 3D structure data for the chemical object, the discrete atom type, and the conditional label into the trained diffusion model comprises inputting the 3D structure data for the chemical object, the discrete atom type, and the conditioning label into a multilayer perceptron.
